(19) United States
(12) Patent Application Publication
(10) Pub. No.: US 2020/0078453 A1
(43) Pub. Date: Mar. 12, 2020

(54) METHODS OF TREATING A MAMMAL SUFFERING FROM OR SUSCEPTIBLE TO AN IMMUNE REACTION TO DRUG TREATMENT

(71) Applicant: Idogen AB, Lund (SE)

(72) Inventors: Peter Ericsson, Lund (SE); Lars Hedbys, Lund (SE); Leif Salford, Lund (SE); Hans-Olov Sjogren, Lund (SE)

(21) Appl. No.: 16/675,988

(22) Filed: Nov. 6, 2019

Related U.S. Application Data
(63) Continuation of application No. 15/271,178, filed on Sep. 20, 2016, now abandoned.
Application No. 15/271,178 is a continuation-in-part of application No. PCT/EP16/56050, filed on Mar. 18, 2016.

(30) Foreign Application Priority Data
Mar. 19, 2015 (GB) 1504701.2

(51) Int. Cl. A61K 39/00; A61K 31/20; C12N 5/0784; A61K 31/7068; A61K 38/21

(52) U.S. Cl. CPC A61K 39/0013; A61K 31/20; C12N 5/064; A61K 31/7068; A61K 39/001; C12N 2501/06; A61K 2039/5154; A61K 2039/577; C12N 2501/71; C12N 2501/02; A61K 38/217

(57) ABSTRACT

There is provided according to the invention a method of treating a mammal suffering from or susceptible to an immune reaction to drug treatment comprising the raising of anti-drug antibodies which method comprises (a) ex-vivo treating antigen presenting cells obtained from the mammal with an agent which induces IDO in said antigen presenting cells in the presence of said drug or an epitope containing fragment thereof and (b) after IDO has been induced in said antigen presenting cells, transferring said cells back to the mammal thereby to establish immune tolerance to the drug.

### RELATED APPLICATIONS

The present application is a Continuation of U.S. application Ser. No. 15/271,178 filed 20 Sep. 2016 which is a Continuation-In-Part of International Application No. PCT/EP16/56050 filed 8 Mar. 2016; which claims priority to United Kingdom Patent Application No. GB1504701.2 filed 19 Mar. 2015; each of which is incorporated herein by reference in its entirety.

### FIELD OF THE INVENTION

The invention relates to a method of treating mammalian subjects who develop anti-drug antibodies, especially antibodies against Factor VIII (FVIII). Specifically, it relates to the use of autologous antigen presenting cells (particularly dendritic cells) treated ex-vivo with an IDO inducing agent (such as zebularine) in the presence of a drug antigen (such as FVIII) for this purpose.

### BACKGROUND OF THE INVENTION

### Anti-Drug Antibodies

Haemophilia A (HA) is an X-chromosome linked bleeding disorder caused by a variety of mutations in the F8 gene encoding FVIII that interfere with the expression or pro-coagulant function of the translated protein. FVIII is expressed primarily in liver and endothelial vascular beds. Lacking sufficient pro-coagulant activity, haemophilia A patients are prone to bleeding episodes and their sequelae, including increased morbidity and mortality. The FVIII database currently identifies 2,015 unique FVIII variants based on 5,472 individual case reports. This vast array of point mutations (66.5%), deletions (23.2%) and others (duplication, polymorphism, insertions, indel and complex) leads to a variety of clinical outcomes. Patients can be treated acutely (on-demand) or prophylactically with either plasma-derived or recombinant FVIII.

A significant number of patients form neutralizing antibodies, termed “inhibitors”, which block the activity of the administered FVIII because their immune systems have not been rendered fully tolerant to certain sequences of normal FVIII. Inhibitor development is currently the most significant treatment complication seen in patients with haemophilia.

At present, once inhibitors form, the only proven method for eradication is immune tolerance induction (ITI) through regular FVIII infusions but this treatment strategy fails in 10-20% of patients.

The problem of anti-drug antibodies is not limited to haemophiliacs with a defective FVIII. Anti-drug antibodies can develop in subjects treated with other drugs.

It is believed that the first step of an immune response against FVIII is recognition of FVIII by antigen presenting cells (APCs). Following endocytosis of FVIII by the APCs, FVIII is processed into small peptides which are then loaded on MHC class II molecules at the cell surface for presentation to FVIII-specific CD4⁺ T cells. The activation of these T cells requires additional signals provided by the APC. These signals are membrane-associated interactions between molecules such as CD40, CD80, CD83 and CD86 on the plasma membrane of the APC and CD28, CD154 and CTLA-4 on the T cell. In combination with these interactions, the APC signals to T cells via cytokines such as IL-12 or IL-10. The combination of signals determines the direction into which the activated T cell differentiates. T helper 1 (Th1) cells generally induce a cytotoxic immune response, Th2 a B-cell mediated antibody response and regulatory T cells are able to induce immunosuppression/tolerance by suppressing B- and T cell responses. Ultimately, these FVIII-specific T cells are able to activate FVIII-specific B-cells and induce affinity maturation and class-switching of immunoglobulin genes in B-cells. As a result, anti-FVIII antibody-secreting plasma cells and circulating FVIII-specific memory B-cells are generated, which are able to produce antibodies upon re-exposure to FVIII.

### Antigen Presenting Cells

Antigen presenting cells (APCs) are cells that display foreign antigens complexed with major histocompatibility complexes (MHCs) on their surfaces; they process antigens and present them to T cells. APCs fall into two categories: professional and non-professional, the professional APC being those that express MHC class II molecules. There are four main types of professional APCs: dendritic cells, macrophages, certain B-cells and certain activated epithelial cells. Interaction of APCs with T-cells occurs in the lymph nodes, to which APCs are directed through the effect of chemotaxis. Dendritic cells can be isolated and matured from a number of sources, such as bone marrow (particularly hematopoietic bone marrow progenitor cells) and blood (particularly peripheral blood mononuclear cells, of which some are immature and are expressing CD34). Depending on the circumstances, (for example, level of expression of co-stimulators such as CD80/86, CD40, or IDO1, PD-L1 or PD-L2 with suppressive activity), the interaction of dendritic cells and T cells in the lymph nodes can lead to an immune or a tolerant response. Thus, a key interaction involves DCs expressing 001 and producing immunosuppressive cytokines (e.g. IL-10 and TGF-β), which induces the T cells to adopt a regulatory phenotype. The regulatory T cells in their turn do not only suppress the effector T cells or naïve T cells in their microenvironment, but they also induce immature DCs to differentiate to tolerogenic rather than immunogenic DCs thereby creating a tolerogenic loop response.

### IDO

IDO1, indoleamine 2, 3-dioxygenase 1, is a haem-containing enzyme catalyzing the initial, rate-limiting step in tryptophan degradation that is known to have an important role in inducing immune tolerance both in experimental animals and in humans. The key role of IDO1 has been clearly identified, for example, during mammalian pregnancy. IDO1 levels are increased and in this way it is essential for induction of immune tolerance vs. the fetus, and experimental inhibition of IDO1 in mice has been shown to result in rejection of fetuses expressing different MHC molecules than their mother.

When intracellularly expressed by DCs or other antigen presenting cells, IDO1 functions as a natural immunoregulator by suppressing T cell responses which results in immune tolerance.

### Zebularine

Zebularine is a cytidine analogue containing a 2-(1H)-pyrimidinone ring which is an effective inhibitor of DNA methylation. Zebularine is very stable with a half-life of approximately 44 h at 37° C. in PBS at pH 1.0 and ˜508 h at pH 7.0, making oral administration of the drug possible. Indeed, orally administered zebularine has been shown to cause demethylation and reactivation of a silenced and hypermethylated p16 gene in human bladder tumour cells grown in nude mice. Zebularine also appears to be minimally cytotoxic in vitro and in vivo, at used concentrations, although toxic at high concentrations. Zebularine is known to induce IDO and the mechanism by which IDO is upregulated by zebularine has been suggested to be through epigenetic upregulation of the IDO1 gene.

WO2008/147283 discloses the use of zebularine for the manufacturing of a medicament for the treatment of an auto-immune disorder or disease or immune rejection of transplants or gene therapeutically modified cells, wherein the treatment induces IDO.

WO2012/087234 discloses a composition comprising at least two compounds, each of which induce indolamine 2,3-dioxygenase (IDO), for the treatment of an autoimmune disorder or disease or suffering from immune rejection of organs, tissues, normal cells or gene therapeutically modified cells, wherein said IDO inducers have different mechanisms of action and give rise to a synergistic effect on the IDO level. It also discloses a method of inducing IDO in a cell culture comprising the steps of (a) providing isolated cells in a suitable medium, (b) adding such a composition, (c) incubating said isolated cells with the composition and (d) obtaining a cell culture in which IDO is induced. It also discloses a method of treating a mammal having an autoimmune disorder or disease or suffering from immune rejection of organs, tissues, normal cells or gene therapeutically modified cells, wherein the treatment induces IDO, comprising firstly a treatment ex vivo of cells derived from the treated mammal or from another mammal, with a therapeutically effective amount of such a composition in the presence of one or more antigens associated with a condition being treated, followed by the transfer of treated cells to the mammal being treated.

Dhodapkar (2001) is a report of antigen-specific inhibition of effector T cell function in humans after injection of immature DCs.

Giannoukakis (2011) is a report of a Phase I (safety) study of autologous tolerogenic DCs in Type 1 diabetic patients.

Nittby (2013) is a report that zebularine induces long term survival of pancreatic islet allotransplants in Streptoxotocin treated diabetic rats.

None of the above prior art documents concern treatments for subjects who develop anti-drug antibodies.

The present invention addresses the need for a method of treating subjects who do not respond to the traditional method of ITI referred to above.

### SUMMARY OF THE INVENTION

The invention provides a method of treating a mammal suffering from or susceptible to an immune reaction to drug treatment comprising the raising of anti-drug antibodies which method comprises (a) ex-vivo treating antigen presenting cells (APCs) obtained from the mammal with an agent which induces IDO in said APCs in the presence of said drug or an epitope containing fragment thereof and (b) after IDO has been induced in said APCs, transferring said cells back to the mammal thereby to establish immune tolerance to the drug.

The invention also provides a method of inducing IDO in a cell culture by a method comprising ex-vivo treating APCs obtained from a mammal with an agent which induces IDO in said APCs in the presence of a drug or an epitope containing fragment thereof as well as APCs in which IDO has been induced obtained by said method.

The invention also provides APCs as aforesaid for use in a method of treating a mammal suffering from or susceptible to an immune reaction to drug treatment comprising the raising of anti-drug antibodies whereby according to said method said cells are transferred back to said mammal thereby to establish immune tolerance to the drug.

Without being limited by theory, according to the method of the present invention, subjects that have developed anti-drug antibodies are treated with autologous APCs that have been treated with zebularine (or other IDO inducer), which skews the phenotype of the APCs towards being tolerogenic. These tolerogenic APCs are primed ex-vivo with corresponding drug antigen and transferred back to the subject. The APCs migrate to the lymph nodes and spleen where they exert their tolerogenic effect upon other immune cells (including the generation of regulatory T cells and B-cells) leading to establishment of tolerance to the drug by initiation of a tolerogenic loop by regulatory T-cells skewing naïve DCs towards tolerogenic differentiation.

### BRIEF DESCRIPTION OF THE FIGURES

FIG. 1 shows the suppressive effects of BMDCs cultured under various conditions on lymphocyte proliferation response to the antigen ovalbumin in vitro (see Example 1)

FIG. 2 shows the suppressive effects of BMDCs cultured under various conditions on T cell proliferation response to the antigen ovalbumin in vitro (see Example 2)

FIG. 3 shows the migration of BMDCs cultured with zebularine and PGE₂ after s.c. inoculation (see Example 3).

FIG. 4 shows the proliferative response to restimulation in vitro of lymph node cells from immunized rats treated with BMDCs cultured under various conditions (see Example 4).

FIG. 5 shows that treatment of BMDCs with zebularine increases their suppressive effects on the proliferative response to restimulation of FVIII-primed CD4⁺ T-cells to FVIII in vitro (see Example 5).

FIG. 6 shows the immunization/treatment schedule performed in Example 6.

FIG. 7 shows the effect of BMDCs that have been incubated with zebularine and human FVIII on the development of inhibitory anti-FVIII antibodies in vivo (see Example 6).

FIG. 8 shows the effect of zebularine, interferon-gamma and a combination of zebularine and interferon-gamma treated human CD34-derived DCs on T-cell proliferative response in vitro (see Example 8).

FIG. 9 shows the effect of zebularine, interferon-gamma and a combination of zebularine and interferon-gamma treated human CD34-derived DCs on the proliferative response of effector T-cells stimulated by beads coated with anti-CD3 and anti-CD28 antibodies in vitro, after a 6-day culture period (see Example 8).

FIG. 10 shows the effect of zebularine, interferon-gamma and a combination of zebularine and interferon-gamma treated human CD34-derived DCs on the proliferative response of naïve T-cells stimulated by beads coated with anti-CD3 and anti-CD28 antibodies in vitro, after a 7-day culture period (see Example 8).

FIG. 11 shows the effect of azacytidine on IDO1 gene expression (mRNA level) in THP-1 cells (see Example 10).

FIG. 12 shows the effect of deoxyazacytidine on IDO1 gene expression (mRNA level) in THP-1 cells (see Example 11).

### DETAILED DESCRIPTION OF THE INVENTION

### Definitions

The term “indolamine dioxygenase” or “IDO” as used herein means IDO1 (indoleamine 2,3-dioxygenase, EC 1.13.11.52) or IDO2 (indoleamine-pyrrole 2,3 dioxygenase-like 1, EC 1.13.11.-) these being two different proteins that can catabolize tryptophan, and can be expressed by APCs.

“Zebularine” means 2′-O-t-Butyldimethylsilyl-3′-O-[(di-isopropylamino)(2-cyanoethoxy)phosphino]-5′-O-(4,4′-dimethoxytrityl)-2(1H)-pyrimidinone-1-β-D-riboside, also known as 1-β-D-Ribofuranosyl)-1,2-dihydropyrimidin-2-one or 2-Pyrimidone-1-β-D-riboside

An analogue is a molecule that differs in chemical structure from a parent compound, for example a homolog (differing by an increment in the chemical structure, such as a difference in the length of an alkyl chain), a molecular fragment, a structure that differs by one or more functional groups, a change in ionization. Structural analogues are often found using quantitative structure activity relationships (QSAR), with techniques such as those disclosed in Remington (The Science and Practice of Pharmacology, 19^{th} Edition (1995), chapter 28).

The term “synergistic effect” in the context of increasing IDO levels after the use of a combination of IDO inducers is intended to mean an increase in the IDO levels that is higher (preferably significantly higher) than the sum of the IDO levels achieved with each of the IDO inducers if used alone, said sum usually being referred to as an “additive effect”.

### Mammals

Mammals that may be treated by methods according to the invention include humans, domestic animals and livestock animals, especially humans. Example domestic animals include cats and dogs. Example livestock animals include horses, cows, sheep and goats.

### APCs

APCs suitable for use in the method of the invention are APCs which express IDO, especially IDO1. Suitably the APCs are capable of inducing a tolerogenic loop, for example, by generating antigen specific regulatory T cells or B-cells.

Most suitably, the APCs are dendritic cells (DCs) such as bone marrow-derived dendritic cells (BMDCs), DCs generated from CD34+ hematopoetic progenitor cells (especially as present in peripheral blood) or DCs derived from peripheral blood mononuclear cells (PBMCs). DCs that are capable of proliferating are preferred. It appears possible to induce DCs derived from peripheral blood mononuclear cells (PBMCs) to proliferate by appropriate treatment (see e.g. Langstein, 1999).

In another aspect of the invention, the APCs are mesenchymal stem cells co-cultured with DCs.

### IDO Inducers

Agents which induce IDO in methods according to the invention include zebularine or an analogue or salt thereof. Examples of zebularine analogues that may be used as IDO inducers according to the invention include 2′-deoxyzebularine, 5-fluoro-zebularine, 5-fluoro-2′-dexyzebularine, 5-chloro-zebularine, 5-chloro-2′-dexyzebularine, 5-bromo-zebularine, 5-bromo-2′-dexyzebularine, 5-iodo-zebularine, 5-iodo-2′-dexyzebularine, 5-methylpyrimidin-2-one, 5-Me-2′-deoxyzebularine, or mono, di or tri phosphates thereof. Examples of salts include salts that may be formed by zebularine with strong organic or inorganic acids such as HBr and HCl salts.

Further agents which induce IDO include other cytidine analogues (e.g. 5-methylcytidine, azacytidine or deoxyazacytidine), histone deacetylase inhibitors (e.g. valproic acid or salts thereof such as sodium valproate, trichostatin A or vorinostate (SAHA)), vitamin D3 analogues (e.g. calcitriol), interferon gamma analogues including interferon-gamma (IFN-gamma), other interferons (e.g. interferon alpha, interferon B1 or interferon-tau), toll like receptor ligands (e.g. CpG containing DNA oligonucleotides or lipopolysaccharides), gonadotropin receptor signalling hormones (e.g. recombinant human gonadotropin (rhCG) or prolactin), prostaglandin E2 analogues, IDO stabilizers (e.g. TGF-b or IL-10), soluble CTLA4 conjugates (e.g. CTLA4-Ig such as abatacept) and glycocorticoids (e.g. dexamethasone). Thus the agent which induces IDO may, for example, be selected from azacytidine and deoxyazacytidine.

In another aspect of the invention, two (or more) agents may be employed which both induce IDO. In one particular embodiment, one of the two IDO inducing agents is zebularine or an analogue or salt thereof. Suitably said two (or more inducers give rise to a synergistic effect and, for example, these two (or more) IDO inducing agents have different mechanisms of action. Said IDO inducers are selected from the group consisting of zebularine or an analogue or salt thereof, other cytidine analogues (e.g. 5-methylcytidine, azacytidine or deoxyazacytidine), histone deacetylase inhibitors (e.g. valproic acid or salts thereof such as sodium valproate, trichostatin A or vorinostate (SAHA)), vitamin D3 analogues (e.g. calcitriol), interferon gamma analogues, other interferons (e.g. interferon alpha, interferon B1 or interferon-tau), toll like receptor ligands (e.g. CpG containing DNA oligonucleotides or lipopolysaccharides), gonadotropin receptor signalling hormones (e.g. recombinant human gonadotropin (rhCG) or prolactin), prostaglandin E2 analogues, IDO stabilizers (e.g. TGF-□ or IL-10), soluble CTLA4 conjugates (e.g. CTLA4-Ig such as abatacept) and glycocorticoids (e.g. dexamethasone). Specific combinations that may be mentioned include (i) zebularine and IFN-gamma, (ii) IFN-gamma and valproic acid, (iii) zebularine, IFN-gamma and valproic acid, (iv) human chorionic gonadotropin (hCG) and zebularine, (v) hCG and IFN-gamma, (vi) zebularine and IFN-A, (vii) zebularine, IFN-gamma and IFN-A, (viii) zebularine, IFN-gamma and TGF-beta, and (ix) zebularine, IFN-gamma, IFN-A, and TGF-beta. Two or more agents that induce IDO may be administered to the cells simultaneously, sequentially or separately (with an appropriate time separation) as will be appropriate for optimum effect.

Obtaining APCs from the Mammal

According to the invention, APCs such as DCs are first obtained from the mammal. One suitable method is to collect immature PBMCs by apheresis (optionally after mobilization of immature cells with standard doses of recombinant G-CSF). Another suitable method is to use Nycodenz centrifugation to separate low density monocytes after lyzing of the red blood cells. To obtain a population of proliferating DCs, immature CD34⁺ cells may be purified from PBMCs using magnetic beads linked to anti-CD34 MAb (Dyna) Biotech, Oslo, Norway). CD34⁺ cell purity can be confirmed by flow cytometry. Purified CD34⁺ cells are typically differentiated into DCs by treatment with GM-CSF and IL-4. Thus, typically they are cultured at 1×10⁵ cells/ml in tissue culture flasks (Nunc, Roskilde, Denmark) in CellGro serum-free medium or RPMI medium containing 10% autologous plasma, 2 mM I-glutamine (JHR), 20-100 ng/ml GM-CSF 200-1000 U/m, IL-4 (R&D Systems, Minneapolis, Minn., USA), optionally together with 5-20 ng/ml IL10 and 50 ng/ml Flt-3L (R&D Systems). Cells may also be cultured with foetal bovine serum or human serum AB. Cultures may be maintained at 37° C. in humidified 5% CO₂ atmosphere for 14 days. As cellular expansion occurs, cell culture medium and cytokines are replenished (e.g. approximately every 4 days).

In some circumstances it may be possible to isolate BMDCs from a bone of the subject. DC cell differentiation can be induced and cells expanded as described above for CD34⁺ cells.

### Ex-Vivo Exposure to IDO Inducer(s) and Antigen

At day 7-14 e.g. at day 12-14 immature DCs may be exposed to the IDO inducer (such as zebularine) or combination thereof, together with an antigen preparation corresponding to the drug. The concentration of the IDO inducer (or combination) and the length of exposure can be selected for optimum IDO induction. A concentration of 1 μM-1 mM e.g. 50-100 μM is exemplary.

The IDO inducer or combination thereof (such as zebularine) and the antigen preparation can be added to the DCs together or separately.

In order to achieve appropriate migration of the DCs to lymph nodes upon administering the cells into the patient, cells are typically treated for approximately 24 h with prostaglandin E2 (at concentration 1-10 μM) to induce required chemotactic receptors. At day 10-17 e.g. day 15-17, DCs may be harvested, suspended in medium and stored frozen in liquid nitrogen until being released for use.

Typically, a quality control step will be performed prior to use. Quality control should include tests for viability, sterility and endotoxin and expression of IDO1. Additionally, expressions of PD-L1, PD-L2, the chemotactic receptor CCR7, level of expression of the costimulators CD80/CD86, CD40, and MHC-II are to be considered. Expression levels of these substances is an indicator that the DCs are tolerogenic.

### Administration of Cells to the Subject

Cells may be administered back to the mammal by various routes e.g. intravenously, subcutaneously or intracutaneously. The medium containing the cells is suitably containing human albumin as a cell-protecting protein. Typically an amount of 3-10×10⁶ cells/dose in 1-10 doses at weekly to bi-weekly intervals is administered. The treatment can be extended until the desired tolerance is achieved.

### Antigen

The antigen preparation corresponding to the drug may contain the drug in whole or part, said part comprising an epitope containing fragment thereof. Generally, a purified antigen will be used. Suitably, the antigen preparation corresponding to the drug resembles as closely as possible the drug which is being administered to the subject. In the case of FVIII, various recombinant drugs are available which are suitable for this purpose including the commercial products ReFacto AF, NovoSeven, Helixate NexGen, Kogenate Bayer, Advate, NovoEight, Nuwiq, Beriate, Beriate P, Feiba, Haemoctin, Hemofil, Monoclate-P, Octanate [LV], Optivate and Recombinate.

### Immune Reactions and Use of Methods of the Invention

Generally, the methods of the invention are suitable for the treatment of mammalian subjects who develop an immune response comprising the raising of anti-drug antibodies to any drug capable of generating said response, for example any biological drug e.g. a protein drug.

Example protein drugs include blood factors (including FVIII or Factor IX), hormones (including insulin and EPO), growth factors (including EGF, IGF, KGF, HGF and FGF), cytokines (e.g. interleukins), enzymes and the like. Further examples include GCSF and analogues e.g. filgrastim and PEGylated versions thereof (such as pegfilgrastim) and interferons (e.g. interferon beta-la). In one preferred aspect of the invention, the drug is FVIII. In another aspect of the invention, the drug is Factor IX.

Biological drugs may contain polysaccharide components.

Further example biological drugs include engineered proteins (such as fusion and chimeric proteins) and recombinant proteins. In some embodiments the drug may be an antibody. The drug may, for example, be a monoclonal antibody, such as a humanized or fully human monoclonal antibody. The drug may also be a protein construct comprising fragments of immunoglobulin. The term antibody includes antibody-drug conjugates and antibody-nanoparticle conjugates as well as PEGylated analogues. In some embodiments the antibody may be a domain antibody including a single light chain antibody or VHH. The drug may consist of an intact light chain immunoglobulin, or a fragment thereof which comprises at least a variable domain and at least part of the light chain constant region or an ScFv. The drug may be free of heavy chain immunoglobulins. Antibodies include anti-TNF-□ monoclonal antibodies, for example, REMICADE™ (infliximab), ENBREL™ (etanercept), HUMIRA™ (adalimumab), CIMZIA® (certolizumab pegol), SIMPONI® (golimumab; ONTO 148) and anti-VEGF antibodies (e.g. AVASTIN™ (bevacizumab) and LUCENTIS™ (ranibizumab). Further examples include HERCEPTIN™ (trastuzumab), RITUXAN™ (rituximab) and SOLIRIS™ (eculizumab).

Methods of the invention are suitable for the treatment of mammalian subjects who develop an immune reaction to a drug including the raising of anti-drug antibodies in a number of drug treatment situations, such as bleeding disorders (haemophilia A and B; respectively deficiency of FVIII and Factor IX), growth factor deficiency (deficiency of EGF, IGF, KGF, HGF, FGF etc), hormone deficiency (deficiency of insulin, EPO), enzyme replacement therapy and inflammatory and auto-immune disorders (anti-TNF-□ monoclonal antibodies).

An extensive list of biological protein therapeutics in clinical development and approved products are disclosed in the 2013 PhARMA report “Biologics”-http://www.phrma.orq/sites/default/files/pdf/biologics2013.pdf—which gives details of 907 biologics targeting more than 100 diseases. This document is incorporated by reference in its entirety. It is considered that the present invention may be used against these as well as other biological therapeutics where an immune response is developed in the treated subject.

### Advantages

The invention is, in at least some embodiments, expected to provide one or more of the following advantages:
Successful raising of immune tolerance to drug, or reduction in immune intolerance to drug, e.g. as measured by reduced anti-drug antibody levels in the blood;
A customised response to drug which is adapted for the individual subject. For example, each haemophilia A patient will have a unique response to native FVIII based on their own FVIII defect and other genetics such as HLA. Thus the autologous approach of the invention is capable of ensuring the subject's APCs become tolerogenic;
Benign effect with low toxicity at required concentrations;
Fairly rapid time to achievement of useful tolerance following initiation of the treatment regime.

### EXAMPLES

### Materials and Methods for Example 1-5 and 7

### Preparation of DCs for Use in Immunisation

Lewis rats are euthanized and femurs and tibia removed. The bones are then disinfected by immersing them in 70% ethanol for 1 min. Both ends of the bones are cut with sterile scissors and the bone marrow flushed and suspended in BMDC-medium (RPMI 1640 supplemented with 2% v/v normal Lewis rat serum, 10 mM HEPES, 100 U/ml penicillin, 100 μg/ml streptomycin and 50 μM 2-mercaptoethanol) using a 5 ml syringe with a 20-gauge needle. After one wash in BMDC-medium red blood cells are lysed and the bone marrow cells are then washed twice in BMDC-medium and passed through a 70-μm cell strainer.

Bone marrow cells (3.5×10⁶) are cultured at 37° C. and 5% CO₂ in T25 cell culture flasks in 5 ml BMDC-medium supplemented with recombinant rat (rr)GM-CSF (5 ng/ml) and rrIL-4 (5 ng/ml). On day 3, 5 ml BMDC-medium supplemented with rrGM-CSF (5 ng/ml) and rrIL-4 (5 ng/ml) is added to each flask. On day 5, the medium and the non-adherent cells are aspirated and replaced with 5 ml fresh BMDC-medium containing rrGM-CSF (5 ng/ml), rrIL-4 (5 ng/ml) and zebularine (50 μM). Also, rrIL-10 (5 ng/ml) may be added here. On day 7, non-adherent cells and semi-adherent cells growing in clusters are harvested by gently tapping or flushing the flasks, centrifuged and dispersed in fresh BMDC-medium containing only rrGM-CSF (2.5 ng/ml) and zebularine (50 μM) and subcultured in fresh T25 culture flasks (2-3.5×10⁶/5 ml). In the case of separating nonadherent BMDCs from semiadherent BMDCs on day 7 (Example 5), nonadherent BMDCs are harvested 3-4 h after start of subculture and transferred to fresh T75 culture flasks. On day 8, prostaglandin E2 (PGE₂) (1-3 μM) may be added to the flasks (for upregulation of chemokine receptor 7 (CCR7) thereby enabling the BMDCs to migrate to lymph nodes). Also, the BMDCs can be pulsed with antigen by addition of the antigen to the flasks. On day 9 the cells are harvested by gently tapping or flushing the flasks. Cells that are going to be inoculated into rats are then extensively washed (2-3 times) in PBS. After washing, the cells (1-5×10⁶) are resuspended in 0.2 ml PBS and inoculated s.c. in the thigh of the rat. BMDCs that are going to be tested in in vitro proliferation response assays are irradiated with 20 Gy before adding to the cultures.

In Examples 1-4, ovalbumin (OVA) was used as a model antigen.

In Example 5, Factor VIII (FVIII) was used as an antigen.

### In Vitro Proliferation Response Assay for Evaluation of Suppressive Effects

Lewis rats were immunized one time at the tail base with 100 μg OVA emulsified in complete Freund's adjuvant (Examples 1-4), or twice with 150 IU/kg recombinant human FVIII with a 2-week-interval (Example 5). Inguinal lymph nodes were isolated from immunized rats seven days after the immunization. A standard proliferation response assay was performed in 96-well plates in a total volume of 150 μl/well (RPMI 1640 supplemented with 10% v/v heat inactivated fetal calf serum, 10 mM HEPES, 100 U/ml penicillin, 100 μg/ml streptomycin and 50 μM 2-mercaptoethanol) using a total lymph node cell population (Example 1 and 4) (100000/well) or using isolated CD4⁺ T cells (50000/well) with irradiated (20 Gy) syngeneic DCs (10000/well (Example 2) or 5000/well (Example 5)) as stimulators. To generate CD4⁺ T cells (≥98% purity), lymph node cells were stained with an antibody cocktail and the CD4⁺ T cells were isolated by negative selection using magnetic beads. To generate syngeneic Lewis DCs, naive spleen cells were isolated, and centrifuged over 14.5% Nycodenz after lyzing of the red blood cells. The low-density spleen cells were isolated from the gradient interface and from those the OX62⁺ DCs were isolated by positive selection using magnetic beads. Primed lymphocytes were stimulated by adding OVA (100 μg/ml) (Examples 1-4) or FVIII (1 μg/ml) (Example 5) to the cultures. Negative controls were set up with medium only (no OVA or FWD.

After 3 days at 37° C. in 5% CO₂, cultures were pulsed for the last 8 h with 0.5 μCi of [3H]thymidine. The cells were then harvested onto glass-fiber filters, and [3H]thymidine incorporation as a measure of T cell proliferation was measured using standard scintillation procedures. Experiments were performed in three or six replicates and the results were expressed in counts per minute (cpm)±standard deviation (SD).

To assess the suppressive effects of the BMDCs on the proliferative response, irradiated (20 Gy), BMDCs (10000/well (Examples 1 and 2) or 5000/well (Example 5)) were added to the proliferation response assay.

BMDCs express the enzyme inducible nitric oxide synthase (iNOS) which has a strong suppressive effect on T cell proliferation. Expression of iNOS in BMDCs is induced by interferon gamma which is released from activated, proliferating T cells. We are not primarily interested in the suppressive effect of iNOS in our experiments. In fact, since the suppressive effect is so powerful we have to block the enzymatic iNOS activity by adding L-NIL (N⁶-(1-iminoethyl)-L-lysine dihydrochloride), a selective inhibitor of iNOS to ensure that the suppression is not only due to iNOS activity.

### Materials and Methods for Example 6

### Immunizations

Male Lewis rats were immunized with Advate (recombinant human FVIII) 50 U/kg+LPS 1 μg/rat. Advate and LPS were mixed and incubated in +4° C. for 18 hrs before immunization. Rats were immunized i.v. in tail vein.

### Treatment of BMDCs In Vitro

Lewis rats were euthanized and femurs and tibia removed. The bones were then disinfected by immersing them in 70% ethanol for 1 min. Both ends of the bones were cut with sterile scissors and the bone marrow flushed and suspended in BMDC-medium (RPMI 1640 supplemented with 2% v/v normal Lewis rat serum, 10 mM HEPES, 100 U/ml penicillin, 100 μg/ml streptomycin and 50 μM 2-mercaptoethanol) using a 5 ml syringe with a 20-gauge needle. After one wash in BMDC-medium red blood cells were lysed and the bone marrow cells were then washed twice in BMDC-medium and passed through a 70-μm cell strainer.

Bone marrow cells were incubated with GM-CSF (5 ng/ml) and IL-4 (5 ng/ml), day 1-7, and GM-CSF (2.5 ng/ml) only, day 8-9, to generate BMDCs. BMDCs were treated with zebularine (50 μM) from day 5-9. BMDCs (10-20×10⁶ cells in 1 ml) were then pulsed with Advate 100 nmol/ml for 2-3 hrs in 37° C. BMDCs were washed three times in PBS before being resuspended in PBS and administered i.v. (2×10⁶ cells/rat).

### Analysis of Inhibitory Anti-Factor VIII Antibodies

The induction of inhibitory anti-Factor VIII antibodies in rat plasma samples was analyzed through a photometric determination of Factor VIII-activity in human plasma (Chromogenix, Coatest SP4 Factor VIII). Diluted (1:1280) plasma samples from immunized/treated rats are pre-incubated with normal human plasma and thereafter the activity of the human Factor VIII is evaluated with a chromogenic determination.

### Materials and Methods for Examples 10 and 11

### Induction of IDO1 in THP-1 Cells

THP-1 cells were harvested from appropriate number of T75 flasks. Cells were counted in a Bürker chamber, stained with 0.4% Trypan Blue. The appropriate number of cells for each experiment were transferred to a new tube and centrifuged at 1200 rpm, 5 min at room temperature (RT). The supernatant was discarded and fresh RPMI with 5% FBS were added to get a cell concentration of 2×10⁵ cells/mL. The compound was added to the five separate wells in 6-well plates such that five different final concentrations were obtained after adding the cells. A control was added to one well in the 6-well plate to which cells were added. 1×10⁶ cells per well were seeded out to achieve a total volume of 5 mL per well. The cells were incubated at 37° C. in 5% CO₂ for 96 hours. After 96 hours the assay was stopped and the cells were lysed for the isolation of RNA.

### RNA Extraction and Quantification of Gene Expression by RT-qPCR

Total RNA was extracted from THP-1 cells using the RNA RNeasy Mini extraction kit (Qiagen), according to the manufacturer's instructions. A SuperScript® VILO™ RT-kit (Thermo Fisher) was used to reverse transcribe 1 μg of total RNA to cDNA and qPCR was performed. The primers used for amplification of the human IDO1 gene were 5″-TTGTTCTCATTTCGTGATGG-3″ (forward; SEQ ID No. 1) and 5″-TACTTTGATTGCAGAAGCAG-3″ (reverse; SEQ ID No. 2). The primers used for amplification of the endogenous reference gene for human HPRT1 were 5″-CTAATTATGGACAGGACTGAAC-3″ (forward; SEQ ID No. 3) and 5″-AGCAAAGAATTTATAGCCCC-3″ (reverse; SEQ ID No. 4). In brief, RT-qPCR was performed in 20 μl reaction, consisting 10 μl PowerUp SYBR Green Master Mix, 0.5 μM of each primer, 4 μl diluted cDNA for amplification of 001 and 1.5 μl diluted cDNA for amplification of HPRT1. An iCycler iQ real-time detection system (Stratagene, Mx3000P, La Jolla, Calif., USA) with the following thermal profile: UDG incubation at 50° C. for 2 min, then denaturation at 95° C. for 5 min, followed by 45 cycles at 94° C. for 15 s, 58° C. for 30 s, and 60° C. for 30 s, was used to perform thermocycling and real-time detection of PCR products. After amplification a melting curve analysis was performed. The RT-qPCR experiments were always run in triplicate. IDO1 was normalised to the geometric means of two HPRT1 reference gene, using the ΔCt method. Within-group comparisons were normalized to one control sample, using the ΔΔCt method.

### Example 1—BMDCs Cultured with Zebularine (with or without PGE₂) Suppress the Proliferative Response of Lymph Node Cells In Vitro

Rats were immunized at the tail base with 100 μg OVA emulsified in complete Freund's adjuvant. Seven days post immunization inguinal lymph nodes were isolated. Lymph node cells (LN cells, total lymph node population) were cultured in 96-well plates (100000 per well) and re-stimulated with OVA (100 μg/ml) or un-stimulated (control, no OVA) for 3 days with or without the addition of irradiated (20 Gy) BMDCs (10000 per well) cultured with zebularine (50 μM), with or without PGE₂. To ensure that the suppressive effect was not only due to iNOS activity, the iNOS inhibitor L-NIL (0.01 mg/ml) was added to some of the wells. After 3 days at 37° C. in 5% CO₂, cultures were pulsed for the last 8 h with 0.5 μCi of [3H]thymidine.

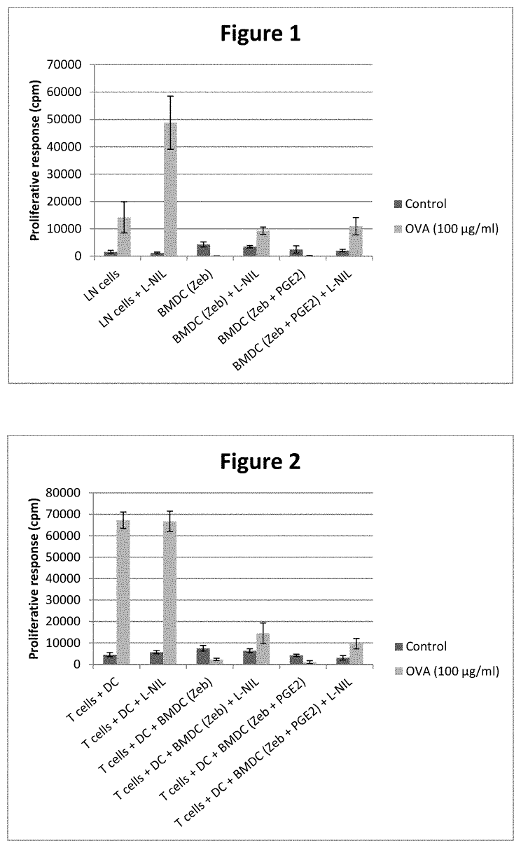

The results are expressed as the mean±SD of sextuplicate wells (FIG. 1).

The results demonstrate a complete suppression of the proliferative response of immune lymph node cells towards the defined antigen ovalbumin when added in vitro in the proportion 1:10. Culture of the DCs in the presence of PGE₂ does not reduce this effect. Based on the limited increase of lymph node cell response in the presence of the iNOS inhibitor L-NIL only a minor part of the effect of the added DCs appears to be due to the expression of the enzyme iNOS that is under other circumstances known to be capable of a strong transient suppression of T lymphocyte reactivity in vitro. The results confirm that the DCs generated do exhibit the capacity to inhibit the response of lymph node cells to protein antigens in vitro.

### Example 2—BMDCs Cultured with Zebularine (with or without PGE₂) Suppress the Proliferative Response of T Cells In Vitro

Rats were immunized at the tail base with 100 μg OVA emulsified in complete Freund's adjuvant. Seven days post immunization CD4⁺ T cells were isolated from inguinal lymph nodes. Also, CD4⁺ T cells (50000 per well) were co-cultured with OX62⁺ DCs (isolated from spleens from untreated control Lewis rats) (10000 per well) in 96-well plates and re-stimulated with OVA (100 μg/ml) or un-stimulated (control, no OVA) for 3 days with or without the addition of BMDCs (10000 per well) cultured with zebularine (50 μM), with or without PGE₂. To ensure that the suppressive effect was not only due to iNOS activity, the iNOS inhibitor L-NIL (0.01 mg/ml) was added to some of the wells. After 3 days at 37° C. in 5% CO₂, cultures were pulsed for the last 8 h with 0.5 μCi of [3H]thymidine.

The results are expressed as the mean±SD of sextuplicate wells (FIG. 2).

These results demonstrate that the admixed DCs are indeed capable of suppressing the response of pure CD4⁺ T lymphocytes (98%, with minimal contamination of B lymphocytes or non-lymphocyte cells). It is particularly important to inhibit those T cells, since they have a central role in executing the immune response of cytolytic effector cells, and helping the antibody producing B-cells to execute the production of antibodies. Note that in this in vitro experiment a type of activating spleen OX62⁺ DCs has to be added to present the antigen for pure T lymphocytes. Our suppressive DCs in this case suppress the activity of these activating DCs.

### Example 3—BMDCs Cultured with Zebularine and PGE₂ Migrate to Draining Lymph Nodes after s.c. Inoculation

Rats were immunized at the tail base with 100 μg OVA emulsified in complete Freund's adjuvant. Five days later rats were inoculated s.c. with 2×10⁶ CFSE-stained BMDCs, cultured with zebularine (50 μM) and PGE₂, in the thighs. Inguinal lymph nodes were isolated 72 h post inoculation of BMDCs and low density lymph node cells were isolated by density centrifugation. Samples from the isolated low density cells were analyzed on a FACSCalibur for detection of CFSE-positive BMDCs.

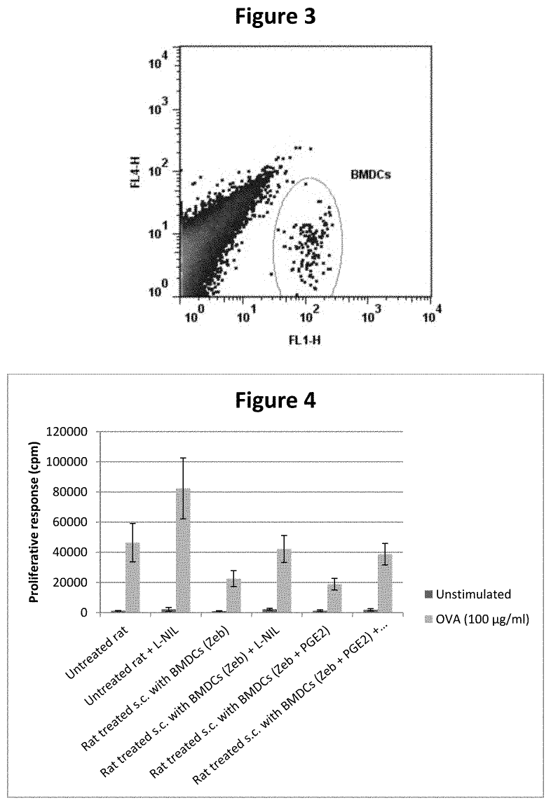

The results are shown in FIG. 3.

An important feature for the in vivo effects of the tolerogenic DCs is that they exhibit the receptors required to be able to migrate to the lymph node draining the area of inoculation. It is the role of PGE₂ treatment of the DCs to enhance the expression of a main receptor (CCR7) of this type. These results show that the suppressive DCs do indeed migrate to the draining lymph node.

### Example 4—Lymph Node Cells from Immunized Rats Treated with BMDCs Cultured with Zebularine (with or without PGE₂) Express Reduced Proliferative Response to Restimulation In Vitro

Rats were immunized at the tail base with 100 μg OVA emulsified in complete Freund's adjuvant. At the same time some of the rats received 2×10⁶ BMDCs cultured with zebularine (50 μM), with or without PGE₂ by s.c. inoculation in the thighs. Seven days post immunization inguinal lymph nodes were isolated. Lymph node cells (LN cells, total lymph node population) were cultured in 96-well plates (100000 per well) and restimulated with OVA (100 μg/ml) for 3 days. To ensure that the suppressive effect was not only due to iNOS activity, the iNOS inhibitor L-NIL (0.01 mg/ml) was added to some of the wells. After 3 days at 37° C. in 5% CO₂, cultures were pulsed for the last 8 h with 0.5 μCi of [3H]thymidine.

The results are expressed as the mean±SD of sextuplicate wells (FIG. 4).

These in vivo results demonstrate that the DCs inhibit the responsiveness of the lymph node lymphocytes when they are inoculated simultaneously with immunization but at a different site although drained to the same lymph node. The inhibitory effect was not due to iNOS activity.

### Example 5—Treatment of BMDCs with Zebularine Increases their Suppressive Effects on the Proliferative Response to Restimulation of FVIII-Primed CD4+ T-Cells to FVIII In Vitro

Rats were immunized twice s.c. at the tail base with 150 IU/kg human FVIII (Advate) mixed with 1 μg LPS with a 2-week-interval. Seven days post immunization CD4⁺ T cells were isolated from inguinal draining lymph nodes. CD4⁺ T cells (50000 per well) were co-cultured with OX62⁺ DCs (isolated from spleens from untreated control Lewis rats) (5000 per well) in 96-well plates and re-stimulated with FVIII (Advate, 1 μg/ml) or un-stimulated for 3 days with or without the addition of semiadherent (“Semiadh”) or nonadherent (“Nonadh”) BMDCs in ratio 1:100 (BMDCs: T cells, 500 BMDCs per well) or 1:10 (BMDCs: T cells, 5000 BMDCs per well) cultured with or without zebularine (“Zeb”) (50 μM). To ensure that the recorded suppressive effect was not only due to iNOS activity, the iNOS inhibitor L-NIL (0.01 mg/ml) was added to some of the wells. After 3 days at 37° C. in 5% CO₂, cultures were pulsed for the last 8 h with 0.5 μCi of [3H]thymidine.

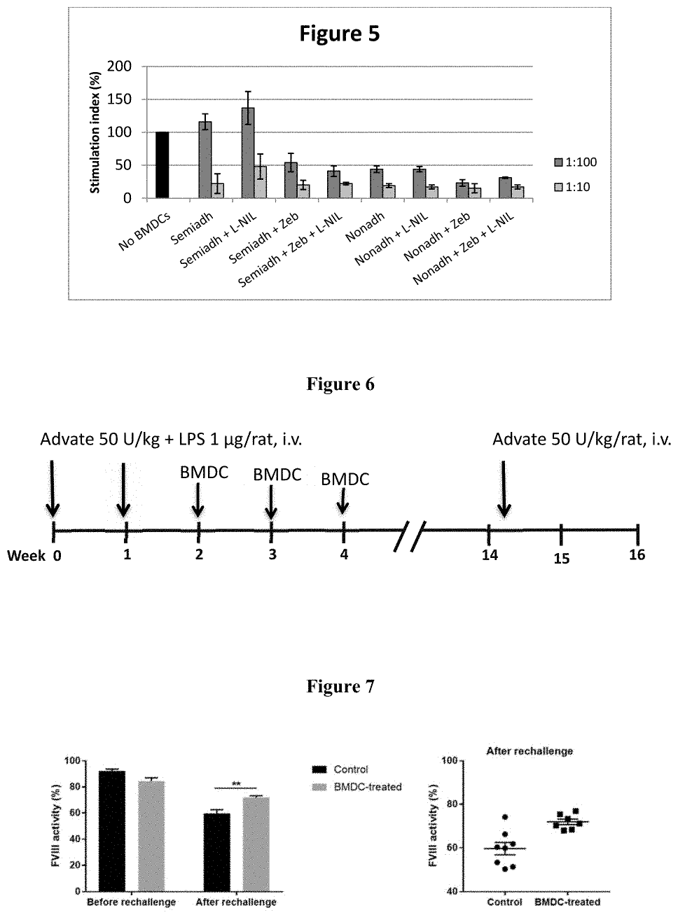

The results are expressed as percent stimulation index. The stimulation index (the FVIII-stimulated proliferative response divided by the non-stimulated proliferative response) was calculated for each sample. The stimulation index for wells without added BMDCs was considered as 100%. The results are expressed as the mean±SD of triplicate wells (FIG. 5).

These results demonstrate that the admixed DCs are indeed capable of suppressing the proliferative response of FVIII-primed CD4⁺ T lymphocytes (with minimal contamination of B lymphocytes or non-lymphocyte cells) to restimulation with FVIII in vitro. Also, these results demonstrate that treatment of DCs with zebularine increases these suppressive effects.

### Example 6—Effect of Zebularine-Treated Dendritic Cells in an Animal Model for the Development of Inhibitory Anti-Factor VIII Antibodies in Hemophilia a

This study was performed in a rat model for the development of inhibitory anti-Factor VIII antibodies in hemophilia A. A modified protocol based on the publication of Jarvis et al. was used for the induction of anti-Factor VIII antibodies. Lewis rats were immunized i.v. with human Factor VIII (50 U/kg/rat) (FVIII; Advate) and LPS (1 μg) twice with one week in-between. Induced immunity towards human FVIII was confirmed by analysis of proliferation response towards human FVIII of T-cells isolated from immunized rats after two weeks. After harvest of bone marrow cells from adult donor rats, differentiation to immature dendritic cells was induced by culturing the cells in GM-CSF and IL-4 as described in Materials and Methods. IDO1-expressing tolerogenic rat dendritic cells are generated in vitro by treatment of the immature dendritic cells with zebularine alone.

BMDCs that had been incubated with zebularine and human FVIII were administered i.v. three times at weekly intervals starting the week after the last Advate immunization. At week 14, the animals were given a re-immunization (also referred to as “re-challenge”) with human FVIII i.v and the study was terminated at week 16. The immunization/treatment schedule is shown in FIG. 6.

The effect of transferring zebularine-treated BMDCs on the development of inhibitory anti-FVIII antibodies was analyzed just before (week 14) and two weeks after re-challenge with Advate (week 16). The amount of neutralizing antibodies towards human FVIII in rat plasma samples was determined as set out in the Materials and Methods section above.

The results are shown in FIG. 7. Incubation of serum from control rats with human plasma containing FVIII resulted in reduced FVIII activity after re-challenge, demonstrating the presence of inhibitory antibodies. On the contrary, serum from rats treated with BMDCs pulsed with zebularine and FVIII showed a significantly lower (P=0.0059) inhibitory effect on FVIII activity upon re-challenge, indicating that the BMDC-treatment reduced the development of inhibitory anti-FVIII antibodies. The effect was observed at week 16, i.e. 12 weeks after the last BMDC-treatment, indicating a long-lasting effect of the tolerogenic BMDCs.

The data show that administration of BMDCs which have been incubated with zebularine and Factor VIII ex vivo reduced the development of inhibitory anti-FVIII antibodies in rat serum.

### Example 7—Suppression of the Immune Response Induced in Rats Immunized with Human FVIII by i.v. Transfer of Dendritic Cells Expressing IDO1 Induced In Vitro

After harvest of bone marrow cells from adult rats, differentiation to immature dendritic cells is induced by culturing the cells in GM-CSF and IL-4. IDO1-expressing tolerogenic rat dendritic cells are generated in vitro by treatment of the immature dendritic cells with zebularine alone or with combinations of IFN-gamma and zebularine or other IDO-inducers (IFN-alpha, valproic acid, TGF-beta).

Adult rats are immunized twice intravenously with 150 IU/kg human FVIII mixed with 1 μg LPS with a 2-week-interval, known to induce both a detectable T-cell and a B-cell immune response to human FVIII. In one group of rats, three doses of in vitro-generated tolerogenic syngeneic DCs loaded with human FVIII are administered i.v., starting one week after last immunization. A control group of rats is similarly immunized but do not receive any i.v. transfer of dendritic cells. Animals are monitored for antibodies to human FVIII by ELISA and for anti-human FVIII T-cell responsiveness by assays for T-cell proliferation (thymidine incorporation assay) and cytokine production by ELISA (IL-2, IL-4, IL-5, IL-17, IFN-gamma, TNF-alpha, and IL-6).

Three doses of IDO1-expressing dendritic cells are expected to suppress T-cell response to human FVIII as assayed in vitro and also to suppress the antibody response to human FVIII. The suppressive effect of the combinations of IFN-gamma and zebularine or another IDO1-inducer is expected to be stronger due to a synergistic effect on the IDO1 induction by the combination of IFN-gamma and the respective inducer. Also the induction of a high affinity transporter of tryptophan in the dendritic cells by IFN-gamma (Bhutia, 2015) should cause a stronger suppressive effect on the immune T-cells that are unable to express this high affinity transporter.

### Example 8—Demonstration In Vitro of a Suppressive Effect of Human Dendritic Cells Treated with IDO-Inducing Agents on the Reactivity of Human T-Cells

CD34⁺ PBMCs were isolated from healthy blood donors. The cells were expanded in vitro and differentiated to immature DCs. The immature DCs were exposed for 3 days to zebularine (60 μM) alone, IFN-gamma alone (60 μM) or a combination of IFN-gamma and zebularine (200 IU/ml and 60 μM respectively) to induce expression of IDO1 and tolerogenic function. Stimulatory DCs were generated in the absence of zebularine and IFN-gamma treatment as the control.

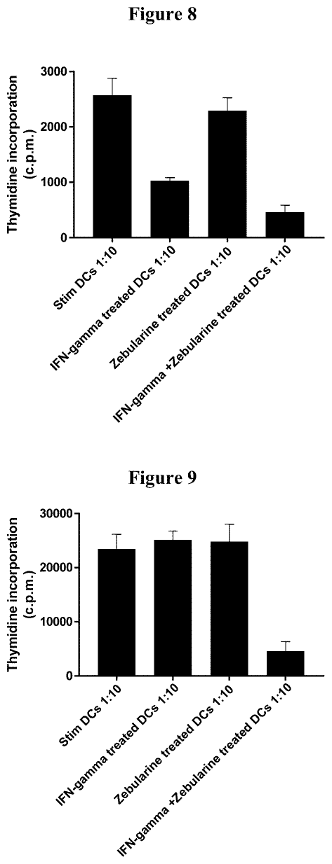

On the day of testing of the generated tolerogenic DCs, CD4+CD45RA⁺ (naïve) or CD4⁺CD45RO⁺ (effector) T-cells were isolated from peripheral blood of a different blood donor and purified (≥95%) by magnetic bead separation. T-cells were then activated in an allogeneic setting with different DC:T-cell ratios of the generated stimulatory or tolerogenic DCs. The T-cell proliferative response was measured by ³H-thymidine incorporation during the final 18 h of a 6-day incubation period and the results are shown in FIG. 8. The treatment was effective at reducing T-cell proliferative response and the use of IFN-gamma in combination with zebularine gave the best result; the low level of thymidine incorporation when the combination of interferon gamma and zebularine was used is indicative of a low T-cell proliferative response meaning that the tolerogenic DCs successfully inhibited the T-cell proliferation and hence, inhibited the production/release of cytokines.

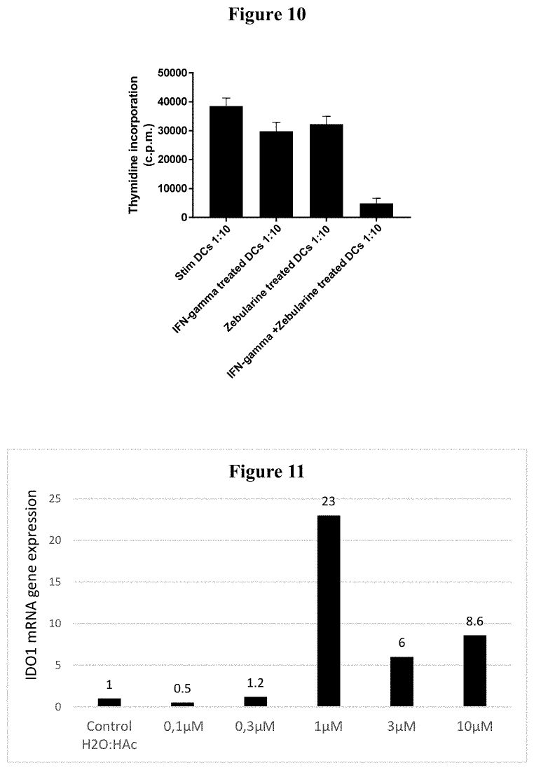

Further experiments were performed whereby the suppressive potential of the tolerogenic DCs on T-cell proliferation was determined by mixing allogeneic T-cells with T-cell activating magnetic beads coated with anti-CD3 and anti-CD28 antibodies, and then titrating in different numbers of stimulatory and tolerogenic DCs. The results are shown in FIGS. 9 and 10. FIG. 9 shows the results following a 6-day culture period of effector T-cells and FIG. 10 shows the results following a 7-day culture period of naïve T-cells. Again, the use of IDO-inducing agents, particularly interferon-gamma in combination with zebularine, resulted in a marked decrease in thymidine incorporation compared to the control (stimulatory DCs), which is indicative of a decrease in T-cell proliferation and production/release of cytokines.

Overall, the generated tolerogenic DCs successfully suppressed the CD4⁺ T-cell proliferative response. This study shows that human white blood cells were successfully reprogrammed to tolerogenic dendritic cells and that by treatment of the DCs with IDO-inducing agents, especially a combination of zebularine and interferon gamma, the DCs inhibit the activation of other immune cells in vitro.

### Example 9—Demonstration In Vitro of a Suppressive Effect of IDO1-Expressing Autologous Dendritic Cells on the Reactivity of T-Cells of an Adult Patient with Haemophilia a Who has Developed FVIII Antibodies (Inhibitors)

CD34⁺ PBMCs are isolated from an adult Haemophilia A patient who has developed antibodies (inhibitors) to FVIII. The cells are expanded in vitro by culture with SCF, Flt3L and differentiated to immature DCs by GM-CSF and IL-4. The immature DCs are loaded with FVIII and exposed for 3 days to zebularine (50 μM) alone, or a combination of IFN-gamma and zebularine or IFN-alpha, or valproic acid, or TGF-beta, respectively, to induce expression of IDO1 and tolerogenic function.

On the day of testing of the generated tolerogenic DCs, autologous CD4⁺ T-cells and monocytes are isolated separately from peripheral blood and purified (≥95%) by magnetic bead separation. T-cell responsiveness of the patient to FVIII in vitro is assayed by exposing 50,000 CD4⁺ T-cells to FVIII at concentration of 0, 0.01, 0.1, 0.3 and 1 μg/ml in the presence of 5,000 monocytes to present the FVIII. The T-cell proliferative response is measured by ³H-thymidine incorporation during the final 8 h of a 3-6 day incubation period. Release of cytokines (IL2, IL4, IL5, IL6, IL17, IFN-gamma, TNF-alpha) to culture medium is assayed by ELISA. Induction of transformation of the T-cells into Tregs by influence of the IDO1-expressing DCs is assayed in FACS by determining frequencies and numbers of FOX-P3⁺ CD4⁺ cells.

The generated tolerogenic DCs are expected to inhibit the CD4⁺ T-cell proliferative response and their production/release of cytokines. Demonstration of a strong inhibition of the Th2 type cytokines will indicate a probable mechanism of a suppressive effect of a B-cell antibody response via suppression of Th2 and will predict that the DCs should have a suppressive effect on the anti-FVIII antibody production in vivo.

### Example 10—Induction of IDO1 in THP-1 Cells with Azacytidine

The procedure under “Induction of IDO1 in THP-1 cells” was followed such that the final concentrations set out in Table 1 were obtained.

TABLE 1
Final concentrations of crystalline 5-azacytidine in 5 mL wells

| Sample No. | Compound | Final Concentration | |
|---|---|---|---|
| 1 | HAc/H₂O 1:1 v/v (control) | 0.5% | v/v |
| 2 | 5-azacytidine | 0.1 | micromolar |
| 3 | 5-azacytidine | 0.3 | micromolar |
| 4 | 5-azacytidine | 1 | micromolar |
| 5 | 5-azacytidine | 3 | micromolar |
| 6 | 5-azacytidine | 10 | micromolar |

Following RNA extraction and quantification of gene expression by RT-qPRC, as described above, induction of IDO1 gene expression using Samples 1 to 6 was investigated.

TABLE 2
Results of 5-azacytidine samples on IDO1 expression

| | Concentration | IDO1 expression |
|---|---|---|
| Sample No. | (micromolar) | (fold change) |
| 1 | control | 1 |
| 2 | 0.1 | 0.5 |
| 3 | 0.3 | 1.2 |
| 4 | 1 | 23 |
| 5 | 3 | 6 |
| 6 | 10 | 8.6 |

The results are also shown in FIG. 11. The results indicate that 5-azacytidine alone increases IDO1 gene expression by up to 23 fold. The highest level of IDO induction was observed at 1 micromolar concentration of 5-azacytidine.

### Example 11—Induction of IDO1 in THP-1 Cells with Deoxyazacytidine

The procedure under “Induction of IDO1 in THP-1 cells” was followed such that the final concentrations set out in Table 3 were obtained.

TABLE 3
Final concentrations of 5-aza-2′-
deoxycytidine (decitabine) in 5 mL wells

| Sample No. | Compound | Final Concentration | |
|---|---|---|---|
| 7 | DMSO (control) | 0.5% | v/v |
| 8 | decitabine | 0.1 | micromolar |
| 9 | decitabine | 0.3 | micromolar |
| 10 | decitabine | 1 | micromolar |
| 11 | decitabine | 3 | micromolar |
| 12 | decitabine | 10 | micromolar |

Following RNA extraction and quantification of gene expression by RT-qPRC, as described above, induction of IDO1 gene expression using Samples 7 to 12 was investigated.

TABLE 4
Results of decitabine samples on IDO1 expression

| | Concentration | IDO1 expression |
|---|---|---|
| Sample No. | (micromolar) | (fold change) |
| 7 | control | 1 |
| 8 | 0.1 | 6 |
| 9 | 0.3 | 16.4 |
| 10 | 1 | 40 |
| 11 | 3 | 87 |
| 12 | 10 | 101 |

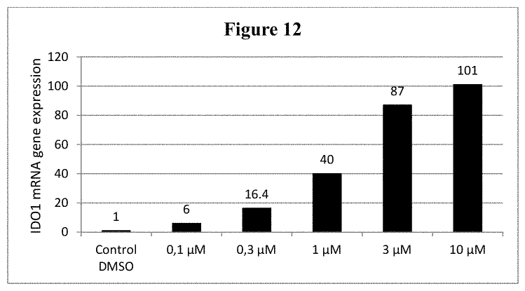

The results are also shown in FIG. 12. The results indicate that 5-aza-2′-deoxycytidine alone increases IDO1 gene expression by up to 101 fold. The highest level of IDO induction was observed at 10 micromolar concentration of decitabine.

### REFERENCES

Dhodapkar, M. et al., J. Exp. Med., 2001, 193(2), 233-238
Giannoukakis, N. et al., Diabetes Care, 2011, 34, 2026-2032
Nittby, H. et al., PLOS ONE, 2013, 8(8), 1-8
Langstein, J. Blood, 1999, 94, 3161-3168
Bhutia, Y. et al., Biochim Biophys Acta, 2015, 1848, 453-462
Jarvis, M. A. et al., Thromb Haemost. 1996, 75, 318-25

### ABBREVIATIONS

### HA Haemophilia A

ITI Immune tolerance induction
DC Dendritic cell
APC Antigen presenting cell
IDO Indolamine dioxygenase
L-NIL N⁶-(1-iminoethyl)-L-lysine dihydrochloride
MHC Major histocompatibility complex

### IFN Interferon

DNA Deoxyribonucleic acid
PBS Phosphate-buffered saline
BMDC Bone marrow-derived dendritic cell
PBMC Peripheral blood mononuclear cell

### OVA Ovalbumin

CFSE Carboxyfluorescein succinimidyl ester

### FVIII Factor VIII

iNOS inducible nitric oxide synthase
s.c. subcutaneous
hCG human chorionic gonadotropin
EGF epidermal growth factor
HGF hepatocyte growth factor
KGF keratinocyte growth factor
TGF tissue growth factor

### TGF-b TGF-beta

TNF tumour necrosis factor
G-CSF granulocyte colony stimulating factor
GM-CSF granulocyte macrophage colony stimulating factor
EPO erythropoietin
Ig immunoglobulin
IFN-A interferon alpha
MAb monoclonal antibody
rr rat recombinant
FACS fluorescence activated cell sorting
TNF-alpha tumour necrosis factor-alpha
Flt3L FMS-like tyrosine kinase 3 ligand
SCF stem cell factor
μ micro
ELISA enzyme-linked immunosorbent assay
Tregs regulatory T-cells

### IL Interleukin

i.v. intravenous
FBS foetal bovine serum
RNA ribonucleic acid
HAc acetic acid
LN lymph node
SD standard deviation

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

All patents and patent applications referred to herein are incorporated by reference in their entirety.

1. A method of treating a mammal suffering from or susceptible to an immune reaction to drug treatment comprising the raising of anti-drug antibodies which method comprises (a) ex-vivo treating antigen presenting cells obtained from the mammal with an agent which induces IDO in said antigen presenting cells in the presence of said drug or an epitope containing fragment thereof and (b) after IDO has been induced in said antigen presenting cells, transferring said cells back to the mammal thereby to establish immune tolerance to the drug.

2. The method according to claim 1 where the mammal is a human.

3. The method according to claim 1 wherein the drug is a biological drug.

4. The method according to claim 3 wherein the drug is FVIII.

5. The method according to claim 1 wherein the antigen presenting cells are dendritic cells.

6. The method according to claim 5 wherein the dendritic cells are bone marrow-derived dendritic cells, dendritic cells generated from CD34+ hematopoietic progenitor cells or dendritic cells generated from peripheral blood mononuclear cells.

7. The method according to claim 1 wherein the agent is an IDO stabilizer.

8. The method according to claim 7 wherein the IDO stabilizer is TGF-b.

9. The method according to claim 1 wherein two or more agents which induce IDO are employed in step (a).

10. The method according to claim 9 wherein one of the two or more agents which induce IDO is TGF-b.

11. A method of inducing IDO in a cell culture by a method comprising ex-vivo treating antigen presenting cells obtained from a mammal with an agent which induces IDO in said antigen presenting cells in the presence of a drug or an epitope containing fragment thereof.

12. The method according to claim 11 wherein the antigen presenting cells are dendritic cells.

13. The method according to claim 11 wherein the drug is a biological drug.

14. The method according to claim 13 wherein the drug is FVIII.

15. The method according to claim 11 wherein the agent is an IDO stabilizer.

16. The method according to claim 15 wherein the IDO stabilizer is TGF-b.

17. The method according to claim 14 wherein the agent is an IDO stabilizer.

18. The method according to claim 17 wherein the IDO stabilizer is TGF-b.

19. A method of treating a mammal suffering from or susceptible to an immune reaction to drug treatment comprising raising anti-drug antibodies wherein said method comprises administering the antigen presenting cells in which IDO has been induced obtained by the method of claim 11 to said mammal thereby to establish immune tolerance to the drug.

20. The method according to claim 19 wherein the antigen presenting cells are dendritic cells.


SEQUENCE LISTING (4 sequences)

SEQ ID NO: 1 - DNA, 20 bp, Homo sapiens
ttgttctcatttcgtgatgg

SEQ ID NO: 2 - DNA, 20 bp, Homo sapiens
tactttgattgcagaagcag

SEQ ID NO: 3 - DNA, 22 bp, Homo sapiens
ctaattatggacaggactgaac

SEQ ID NO: 4 - DNA, 20 bp, Homo sapiens
agcaaagaatttatagcccc